(19) United States
(12) Patent Application Publication
(10) Pub. No.: US 2003/0113266 A1
(43) Pub. Date: Jun. 19, 2003

(54) Material for evaluating dental caries activity

(75) Inventor: Yuko Matsumoto, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: GC CORPORATION, Tokyo (JP)

(21) Appl. No.: 10/299,800

(22) Filed: Nov. 20, 2002

(30) Foreign Application Priority Data
Dec. 14, 2001 (JP) 2001-381545

(51) Int. Cl. A61K 49/00; A61K 7/16

(52) U.S. Cl. 424/9.71; 424/49

(57) ABSTRACT

A material for evaluating dental caries activity that is capable of evaluating dental caries activity, i.e., the released amount of an acid from dental plaque, which is important in dental health, by a simple manner in a short period of time, contains an absorptive material having carried thereon a pH indicator having an indicator range of pH 3.5 to 8.0 and a sugar, and has been adjusted to have a pH value higher than the indicator range of the pH indicator, in which dental plaque taken from a subject is directly applied on the material to evaluate dental caries activity by change in color.

### BACKGROUND OF THE INVENTION

1. Field of the Invention

The present invention relates to a material for evaluating dental caries activity, which is capable of evaluating activity of dental caries in an oral cavity of a subject by a simple manner in a short period of time.

2. Description of Conventional Art

Evaluation of dental caries activity in dental surgery is to estimate and determine activity of dental caries, i.e., as to whether or not dental caries of teeth currently developed further proceeds, and as to whether or not there is a possibility of future development of dental caries due to activity of dental caries although no dental caries is currently developed. Therefore, it has great significance in dental health.

It is considered that development and progress of dental caries of teeth are caused in such mechanisms referred to as a decalcifying phenomenon that cariogenic bacteria present in dental plaque attached to the teeth metabolize carbohydrate to produce an acid, and calcium ions and phosphoric ions in the teeth are eluted by the acid. Therefore, a method of measuring a released amount of an acid from dental plaque is studied as a method for evaluating dental caries activity. Because it is considered that the released amount of an acid from dental plaque depends on the number of cariogenic bacteria present in the dental plaque, such a method is carried out by a measuring test for the number of mutans streptococcus in the dental plaque and a measuring test for the number of lactobacilli in the dental plaque. However, these methods have such disadvantages that expensive culturing equipments, sophisticated operation techniques and prolonged culturing periods are required.

Under the circumstances, various methods capable of evaluating in simple manners have been proposed, and for example, they are disclosed in JP-A-50-1589, JP-A-54-47700, JP-A-56-96700, JP-A-56-120623, JP-B-57-13824 and JP-A-59-99354. These methods each include such a process that dental plaque taken from teeth of a subject is added to an aqueous solution containing a pH indicator and a sugar, and the released amount of an acid is then evaluated by the change in color shown by the pH indicator in the aqueous solution. These are excellent in smipleness in comparison to the conventional method for measuring number of bacteria because the dental caries activity can be evaluated only by adding the dental plaque into the aqueous solution.

However, these methods have such disadvantages that because it is necessary to add the dental plaque to the aqueous solution, the dental plaque is dispersed in the aqueous solution to dilute the acid produced by the cariogenic bacteria in the dental plaque, and therefore, a prolonged period of time from the addition of the dental plaque, such as 30 minutes to 48 hours, is required for the change in color shown by the pH indicator and thus a long period of time is required for the evaluation.

### SUMMARY OF THE INVENTION

An object of the present invention is to solve the problems associated with the conventional techniques and to provide a material for evaluating dental caries activity, which is capable of evaluating activity of dental caries of a subject, i.e., a released amount of an acid from dental plaque, by a simple manner in a short period of time.

As a result of earnest investigations made by the inventors for solving the problems, it has been found that when dental plaque taken from a subject is directly applied on a material for evaluating dental caries activity that contains an absorptive material having carried thereon a pH indicator having an indicator range of pH 3.5 to 8.0 and a sugar and has been adjusted to have a pH value higher than the indicator range of the pH indicator, an acid developed by the dental plaque is not diluted, but the pH indicator sensitively responds thereto in a short period of time to cause change in color. Accordingly, the evaluation can be carried out by observation of change in color in a short period, since it is not necessary to observe change in color of the pH indicator after cultivation consuming a long period of time. Thus, the present invention has been completed.

The present invention relates to a material for evaluating dental caries activity containing an absorptive material having carried thereon a pH indicator having an indicator range of pH 3.5 to 8.0 and a sugar, and having been adjusted to have a pH value higher than the indicator range of the pH indicator, in which dental plaque taken from a subject is directly applied on the material to evaluate dental caries activity by change in color. In the present invention, it is preferred that the pH indicator is at least one selected from the group consisting of p-ethoxychrysoidine, α-naphthyl red, alizarine sodium sulfonate, bromo cresol green, methyl red, 2,5-dinitrophenol, p-nitrophenol, azolitmin, bromo cresol purple, bromo phenol red, chloro phenol red, phenol red, m-nitrophenol, neutral red, rosolic acid, bromo thymol blue, china blue, lacmoid and resazurin, and it is also preferred that the sugar is at least one selected from the group consisting of glucose, sucrose, fructose, lactose, maltose, an isomerized sugar, isomaltooligosaccharide, panoseoligosaccharide, starch syrup and a coupling sugar.

### DESCRIPTION OF THE PREFERRED EMBODIMENTS

The absorptive material used in the material for evaluating dental caries activity of the present invention is not particularly limited as far as it can carry the pH indicator, the sugar and an alkali solution for maintaining the pH value higher than the indicator range of the pH indicator. Paper, such as filter paper, absorbent paper and paper towel, is most preferred, and also cloth and nonwoven cloth formed with fibers, such as absorbent cotton, silica wool, glass wool, wool, silk, cotton, linen, acrylic fibers, rayon, nylon, nitrocellulose, cellulose acetate, regenerated cellulose and glass fibers, can also be used. Furthermore, a material formed by molding dextran, mutan, levan or cellulose powder can also be used. The form of the absorptive material is preferably a sheet having a thickness of 0.2 to 2 mm and in a substantial square planar shape with an edge length of 0.3 to 30 cmor a substantial circular planar shape with a diameter of 0.3 to 30 cm, more preferably at operatability a sheet square planar shape with an edge length of 0.3 to 8 cm or a sheet circular planar shape with a diameter of 0.3 to 8 cm and more preferably a sheet with an edge length of 0.3 to 2 cm or a sheet with a diameter of 0.3 to 2 cm is suitable for applying a small amount of dental plaque, which is difficult to be taken in a large amount.

The pH indicator is not particularly limited as far as it has an indicator range in a range of pH 3.5 to 8.0, and examples thereof include p-ethoxychrysoidine (indicator range: 3.8 to 5.4), α-naphthyl red (indicator range: 3.7 to 5.0), alizarine sodium sulfonate (indicator range: 5.5 to 6.8), bromo cresol green (indicator range: 3.8 to 5.9), methyl red (indicator range: 4.4 to 6.2), 2,5-dinitrophenol (indicator range: 4.0 to 5.8), p-nitrophenol (indicator range: 5.0 to 7.0), azolitmin (indicator range: 5.0 to 8.0), bromo cresol purple (indicator range: 5.2 to 6.8), bromo phenol red (indicator range: 5.2 to 7.0), chloro phenol red (indicator range: 5.2 to 6.8), phenol red (indicator range: 6.4 to 8.0), m-nitrophenol (indicator range: 6.4 to 8.8), neutral red (indicator range: 6.8 to 8.0), rosolic acid (indicator range: 6.8 to 8.0), bromo thymol blue (indicator range: 6.0 to 7.6), china blue (indicator range: 2.8 to 8.0), lacmoid (indicator range: 4.4 to 6.4) and resazurin (indicator range: 3.8 to 6.5). These pH indicators may be used by mixing two or more kinds of them. The concentration of the pH indicator carried on the absorptive material is preferably about 0.001 to 0.5% by weight, and more preferably 0.005 to 0.03% by weight, based on the weight of the dry absorptive material, because when it is too small, the change in color is unintelligible, whereas when it is too large, a prolonged time is required for the change in color, and the color tone becomes dark to make the change in color unintelligible.

The sugar to be carried on the absorptive material is not particularly limited as far as cariogenic bacteria can produce an acid through metabolism therewith, and usable examples thereof include glucose, sucrose, fructose, lactose, maltose, an isomerized sugar, isomaltooligosaccharide, panoseoligosaccharide, starch syrup and a coupling sugar. The concentration of the sugar carried on the absorptive material is preferably about 1.0 to 50% by weight, and more preferably 5 to 25% by weight, based on the weight of the dry absorptive material, because when it is too small, the pH indicator does not suffer change in color in a short period of time due to a small amount of an acid formed by cariogenic bacteria, whereas when it is too large, cariogenic bacteria receive an osmotic pressure to affect development of an acid.

The material for evaluating dental caries activity according to the present invention can be obtained in such a manner that the pH indicator and the sugar are dissolved in a solvent having no influence on cariogenic bacteria and no buffering function, such as water, which is adjusted to have a pH value higher than the indicator range of the pH indicator with a sodium hydroxide aqueous solution or a potassium hydroxide aqueous solution depending on necessity, and the absorptive material is immersed in or sprayed with the resulting solution. The pH value is preferably higher than such a value that is higher than the estimated pH value of dental plaque.

Upon evaluating dental caries activity by using the material for evaluating dental caries activity according to the present invention, dental plaque of a subject is taken and directly applied on the material for evaluating dental caries activity, and change in color shown by the material for evaluating dental caries activity is observed after a prescribed period of time has lapsed. At this time, cariogenic bacteria present in the dental plaque applied on the material for evaluating dental caries activity metabolize the sugar contained in the absorptive material to produce an acid, and the pH of the absorptive material is lowered by the acid beyond the indicator range of the pH indicator to change the color of the absorptive material, which is then observed. The color shown by the absorptive material after a prescribed period of time has lapsed differs depending on the amount of the acid produced through metabolism of the sugar by cariogenic bacteria, and the amount of the acid produced depends on the number of cariogenic bacteria present in the dental plaque. Therefore, the dental caries activity of the subject can be evaluated by observing the color shown by the material for evaluating dental caries activity after the prescribed period of time has lapsed. As examples of the change in color from a higher pH value to a lower pH value of the pH indicator used, resazurin shows a color changing from blue, magenta to pink, lacmoid shows a color changing from blue, magenta to pink, methyl red shows a color changing from yellow, pink to red, bromo thymol blue shows a color changing from blue, green to yellow, and bromo cresol purple shows a color changing from purple, greenish yellow to yellow.

### EXAMPLES

The present invention will be described in more detail with reference to the following examples, but the invention is not construed as being limited thereto.

Production of Material for Evaluating Dental Caries Activity

Filter paper in a strip form having a width of 10 mm, a length of 10 mm and a thickness of 0.38 mm (Filter Paper 3, a trade name, produced by WHATMAN Inc.) was prepared as the absorptive material, and it was immersed in the aqueous solutions (1) to (4) prepared in the compositions shown in Table 1 below and then taken out therefrom, followed by drying. The resulting pieces of paper were designated as materials for evaluating dental caries activity 1 to 4. The aqueous solutions (1) to (4) were adjusted to have pH 7.2 by using a 0.1N sodium hydroxide aqueous solution.

### Example 1

About 1 mg each of dental plaque taken from two subjects A and B was applied on the materials for evaluating dental caries activity 1 to 4. The materials were allowed to stand in an atmosphere at 37° C. for 5 minutes, and the colors shown by the materials for evaluating dental caries activity were observed.

The results obtained are shown in Table 1.

TABLE 1

| | | | Aqueous | Aqueous | Aqueous | Aqueous |
|---|---|---|---|---|---|---|
| | | | solution 1 | solution 2 | solution 3 | solution 4 |
| Aqueous | Concent- | Bromo cresol | 0.003% by weight | — | — | — |
| solution | ration | purple | | | | |
| | | Resazurin | — | 0.005% by | 0.005% by weight | — |
| | | | | weight | | |
| | | Methyl red | — | — | 0.003% by weight | — |
| | | Bromo thymol | — | — | — | 0.003% by |
| | | blue | | | | weight |
| | | Sucrose | 5% by weight | — | 10% by weight | 5% by weight |
| | | Glucose | — | 10% by weight | — | — |
| | | Lactose | — | — | — | 15% by weight |
| | | Distilled water | balance | balance | balance | balance |
| Material | pH | Bromo cresol | 0.006% by weight | — | — | — |
| for | indicator | purple | | | | |
| evaluating | | (purple > | | | | |
| dental | | greenish yellow | | | | |
| caries | | > yellow) | | | | |
| activity | | Resazurin | — | 0.01% by | 0.01% by weight | — |
| | | (blue > magenta | | weight | | |
| | | > pink) | | | | |
| | | Methyl red | — | — | 0.006% by weight | — |
| | | (yellow > pink > | | | | |
| | | red) | | | | |
| | | Bromo thymol | — | — | — | 0.006% by |
| | | blue | | | | weight |
| | | (blue > green > | | | | |
| | | yellow) | | | | |
| | Sugar | Sucrose | 10% by weight | — | 20% by weight | 10% by weight |
| | | Glucose | — | 20% by weight | — | — |
| | | Lactose | — | — | — | 30% by weight |

| | | | | | | | | | |
|---|---|---|---|---|---|---|---|---|---|
| Evaluation results of | Subject | A | B | A | B | A | B | A | B |
| dental caries | Color before | purple | purple | blue | blue | yellow | yellow | blue | blue |
| activity | test | | | | | | | | |
| | Color after | yellow | green- | pink | magenta | red | pink | yellow | green |
| | lapsing 5 min | | ish | | | | | | |
| | | | yellow | | | | | | |

Water absorbing amount of filter paper: ca. 50 mg, weight of filter paper: 25.3 mg

It was understood from the results that the dental caries activity of the subject A is higher than the dental caries activity of the subject B. As results of an actual streptococcal number measurement of the mutans streptococcus in about 1 mg of dental plaque, it was 1×10⁶ CFU for the subject A and 1×10⁵ CFU for the subject B. The evaluation was carried out by the comparison between the subjects in the example. However, the material for evaluating dental caries activity according to the present invention can accurately evaluate the activity of dental caries with a simple operation without relative evaluation, by providing a material for evaluating dental caries activity under prescribed conditions and by previously determining the relationship between the number of cariogenic bacteria in the dental plaque and the pH caused by the activity of bacteria.

As described in the foregoing, by using the material for evaluating dental caries activity according to the present invention, dental caries activity, i.e., the released amount of an acid from dental plaque, which is important in dental health, can be evaluated by a simple manner in a short period of time. Furthermore, the potency in evaluation of dental caries activity thereof can be variously changed by kinds and amounts of the pH indicator and the sugar used and by the pH value adjusted to a higher value than the indicator range of the pH indicator. Thus, the present invention contributes the dental field to exert a significant value.

What is claimed is:

1. A material for evaluating dental caries activity comprising an absorptive material having carried thereon a pH indicator having an indicator range of pH 3.5 to 8.0 and a sugar, and having been adjusted to have a pH value higher than the indicator range of the pH indicator, in which dental plaque taken from a subject is directly applied on the material to evaluate dental caries activity by change in color.

2. A material for evaluating dental caries activity as claimed in claim 1, wherein the pH indicator is at least one selected from the group consisting of p-ethoxychrysoidine, α-naphthyl red, alizarine sodium sulfonate, bromo cresol green, methyl red, 2,5-dinitrophenol, p-nitrophenol, azolitmin, bromo cresol purple, bromo phenol red, chloro phenol red, phenol red, m-nitrophenol, neutral red, rosolic acid, bromo thymol blue, china blue, lacmoid and resazurin.

3. A material for evaluating dental caries activity as claimed in claim 1 or 2, wherein the sugar is at least one selected from the group consisting of glucose, sucrose, fructose, lactose, maltose, an isomerized sugar, isomaltooligosaccharide, panoseoligosaccharide, starch syrup and a coupling sugar.
